(19) United States
(12) Patent Application Publication
(10) Pub. No.: US 2024/0074957 A1
(43) Pub. Date: Mar. 7, 2024

(54) HAIR TREATMENTS

(71) Applicant: Actera Ingredients, Inc., Newtown, PA (US)

(72) Inventors: Daniel Winn, Princeton, NJ (US); Maggie Zakkak, Ewing, NJ (US)

(73) Assignee: Actera Ingredients, Inc., Newtown, PA (US)

(21) Appl. No.: 18/506,806

(22) Filed: Nov. 10, 2023

(51) Int. Cl. A61K 8/362; A61Q 5/00; A61Q 5/02; A61Q 5/12

(52) U.S. Cl. CPC A61K 8/362; A61Q 5/002; A61Q 5/02; A61Q 5/12; A61K 2800/524

(57) ABSTRACT

Various compositions and methods for treating or preventing damage to hair, especially in over-processed or chemically treated hair, are described. The various compositions comprise itaconic acid, or a salt, ester, or anhydride thereof, and are applied in the absence of any oxidizing agent.

### FIELD OF THE INVENTION

The present invention relates to various compositions and methods for treating or preventing damage to hair, especially in over-processed or chemically treated hair.

### BACKGROUND OF THE INVENTION

Human hair is frequently subjected to chemical processes that damage the structural integrity of the hair fiber. Hair is formed from layers of keratin protein which are polymeric, as well as the natural pigments that provide the inherent natural hair color. Due to various beauty and fashion trends, as well as the desire to avoid grey hair, consumers frequently subject their hair to harsh chemical processes such as application of chemicals that are highly reactive and initiate the breaking of intramolecular bonds within keratin, or the destruction of melanin, or both. Popular beauty treatments that involve chemical hair processes include oxidative hair lightening and oxidative hair coloring, during which oxidizing agents such as peroxides, persulfates, and oxidizing enzymes are applied to the hair. Other popular beauty treatments that involve chemical processes are permanent waves and hair relaxers and straighteners, which involve the application of reducing agents such as formaldehyde, lye, and thiol reducing agents such as thioglycolates. These beauty treatments are referred to interchangeably as chemical processes or chemical treatments.

Because hair constantly grows, new hair growth must be treated to match previously treated hair. Therefore, chemical treatments are often performed several times per year, year after year. Hair that repeatedly undergoes chemical treatments is referred to as over-processed or damaged hair.

Hair damage occurs in a number of ways. The oxidizing and reducing agents react with and damage the cuticle which is the exterior layer of dead cells and proteins protecting the cortex of keratin in the hair fiber. The degraded cuticle results in more penetration of oxidizing or reducing chemicals. The damaged cuticle also results in daily assault from environmental UV light, ozone, and moisture all of which further compromise the structural integrity of the hair fiber. Chemical processes also degrade melanin, creating voids in the hair fiber where moisture can diffuse into. Hair fibers, once damaged and with more voids, permit greater diffusion of materials into and out of the hair. Chemical treatment agents penetrate further into the cortex where they react with keratin protein fibrils. Intramolecular keratin bonds are broken, and the keratin proteins degrade into low molecular weight keratin peptide fragments, which are more water soluble. With repeated hair washing the peptide fragments are washed away creating even more porous hair. Over-processed hair becomes increasingly porous with less keratin content compared to virgin untreated hair, has weakened intramolecular bonds, is mechanically compromised, and easily breaks. This damage often results in dissatisfactory feel and appearance in the form of breakage, dullness, limpness, brittleness, flyaway, split ends, tangling, and difficult to manage strands.

Standard treatment for ameliorating the condition of over-processed hair is to apply topical conditioning products such as masks, serums, and conditioners to the hair on a regular basis to return hair to a soft, smooth, more manageable state. Typical conditioning agents include hydrophobic fluids, oils, cationic surfactants or polymers, and silicones. These agents work by adhering to the hair strands and creating a smooth film that gives the feel and appearance of healthy hair. Recently, trends have been leading consumers away from silicones and other film formers, which can leave a heavy-feeling residue on the hair and cause buildup over time, and towards more lightweight products.

Other solutions to treat over-processed hair have been suggested. For example, U.S. Pat. No. 3,472,243 describes applying to damaged hair vinyl monomers, along with oxidizing agents to polymerize the monomer inside the hair fiber. Also, U.S. Pat. No. 3,634,022 describes applying olefinicaly unsaturated polymerizable monomers and an effective amount of a peroxide initiator to hair to yield improved hair setting and hair conditioning benefits.

However, these solutions suffer from several disadvantages. One disadvantage of these compositions and methods is that the in situ polymerization of vinyl monomers produces only polyvinyl polymers, such as polyacrylates. Polyvinyl polymers are stiff fixatives commonly used in coatings and hair sprays. Hair treated with such compositions are stiff and lack the natural bounce and flow of virgin human hair. Furthermore, polyvinyl polymers have only a carbon-carbon backbone, unlike natural keratin which consists of many amide bonds. The existence of polyvinyl polymers inside of hair creates different attractive and repulsive forces compared to the native proteins of hair. Thus, when hair dyes are applied, the hair may not retain the dye in the same manner as natural keratin fibrils.

Alternatively, U.S. Pat. No. 11,273,120 describes a composition and method to repair the hair fiber from the inside that requires an oxidative agent. Requiring an oxidative agent makes at home use less accessible, necessitating the need for salon use and expertise. Nonoxidative processes make hair repair more accessible for at home use allowing for consumers to address the pain points of damaged hair without professional assistance.

There remains, however, a continuing desire amongst consumers and cosmetic manufacturers to further improve hair products, increasing and improving the ability to repair the hair fiber from the inside, to fill voids within fibers, to reduce the porosity of the hair fiber, and to increase the hydrophobicity and strength of fibers within the internal hair cortex without the need for an oxidative agent. By strengthening the internal cortex, hair can withstand repeated chemical oxidation and reduction treatments and maintain structural integrity of the hair fibers.

The present inventors have set out to provide a new composition that can alleviate signs of hair damage utilizing itaconic acid, a naturally occurring unsaturated dicarboxylic acid. Compositions containing itaconic acid decreases flyaway, improve manageability, and provide a softer and smoother feel and appearance of hair, and surprisingly achieve this effect without the presence of an oxidizing agent.

### BRIEF SUMMARY OF THE INVENTION

Various aspects of the present invention are directed to compositions for treating hair (e.g., damaged hair, over-processed hair, and/or chemically treated hair). In various embodiments, the composition comprises itaconic acid or a salt, ester or anhydride thereof utilized as an active ingredient within various hair products in the absence of oxidizing agents.

Further aspects are directed to processes for preparing various compositions as described herein. In various embodiments, the processes comprise combining (a) itaconic acid or a salt, ester, or anhydride thereof (b) a rinse off or leave-in hair product including but not limited to hair mask, conditioner, shampoo, leave-in conditioner, or serum.

Further aspects are directed to methods of treating hair fibers comprising contacting the hair fibers with a hair treatment composition comprising itaconic acid, or a salt, ester, or anhydride thereof, in the absence of any oxidizing agent. In these and other embodiments, the hair fibers are bleached hair fibers that are treated to reduce flyaway, wherein the treatment results in at least about 10% reduction in flyaway as compared to bleached hair fibers not treated with a composition containing itaconic acid, or a salt, ester, or anhydride thereof.

Other objects and features will be in part apparent and in part pointed out hereinafter.

### DETAILED DESCRIPTION OF THE INVENTION

In general, the present invention is directed to hair treatment compositions and methods for treating hair. In particular, various aspects relate to compositions for treating or preventing damage to hair, especially in over-processed or chemically treated hair. Applicants have discovered that itaconic acid, salts, esters and anhydrides thereof, when added to hair products and applied to hair in the absence of oxidizers, can improve feel and appearance of damaged hair. The surprising discovery that itaconic acid, salts, esters and anhydrides thereof can provide specific repair of hair fibers in the absence of these oxidizers is described herein.

### Compositions

The instant disclosure is directed to compositions for treating hair comprising itaconic acid or salt, ester or anhydride thereof. The content of the itaconic acid or salt, ester, or anhydride thereof in the hair treatment compositions can be about 0.1% to about 10% by weight, about 0.5% to about 5% by weight, or about 1.5% to about 4% by weight. The compositions exhibit increased stability when the pH of the composition is from 4.0 to 6.5.

The composition can be formulated in various suitable forms including, for example, low to moderate viscosity liquids, lotions, milks, mousses, sprays, gels, creams, shampoos, conditioners, and the like. In various embodiments, the compositions described herein are formulated as a hair conditioner or shampoo. The hair conditioner can be rinse-off or leave-on conditioner.

The compositions described herein may further comprise one or more additives (e.g., cosmetically acceptable ingredients). Examples of cosmetically acceptable ingredients are those listed in the International Cosmetic Ingredient Dictionary and Handbook and those listed in the United States Pharmacopeia. Cosmetically acceptable ingredients include, but are not limited to preservatives, antioxidants, chelating agents, sunscreen agents, vitamins, dyes, hair coloring agents, proteins, amino acids, natural extracts such as plant extracts, humectants, fragrances, perfumes, oils, emollients, lubricants, butters, penetrants, thickeners, viscosity modifiers, polymers, resins, hair fixatives, film formers, surfactants, detergents, emulsifiers, opacifying agents, volatiles, propellants, liquid vehicles, carriers, salts, pH adjusting agents (e.g., citric acid), neutralizing agents, buffers, hair conditioning agents, anti-static agents, anti-frizz agents, anti-dandruff agents, absorbents, and combinations thereof.

For example, surfactants include various anionic, cationic, nonionic, and amphoteric surfactants. Anionic surfactants include, but are not limited to, those containing carboxylate, sulfonate and sulfate ions. Examples of anionic surfactants include sodium, potassium, ammonium of long chain alkyl sulfonates and alkyl aryl sulfonates. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine. Emollients include, for example, silicone compounds, polyols (e.g., propanediol), and triglycerides.

Emulsifiers include, but are not limited to, copolymers of an unsaturated ester and styrene sulfonate monomer, cetearyl alcohol, glyceryl ester, polyoxyethylene glycol ether of cetearyl alcohol, stearic acid, polysorbate-20, ceteareth-20, lecithin, glycol stearate, polysorbate-60, polysorbate-80, and combinations thereof.

Preservatives include, but are not limited to, glycerin containing compounds, benzyl alcohol, parabens, sodium benzoate, ethylenediamine-tetraacetic acid (EDTA), potassium sorbate, and so on. Antioxidants include, for example, tocopheryls, BHT, ascorbic acid, Camellia sinensis leaf extract, ascorbyl palmitate, magnesium ascorbyl phosphate, carotenoids, resveratrol, triethyl citrate, arbutin, kojic acid, tetrahexydecyl ascorbate, superoxide dismutase, zinc, sodium metabisulfite, lycopene, ubiquinone, and combinations thereof.

Conditioning agents include, for example, silicone-based agents, panthenol, hydrolyzed wheat and/or soy protein, amino acids, rice bran wax, meadowfoam seed oil, mango seed oil, grape seed oil, jojoba seed oil, sweet almond oil, hydroxyethyl behenamidopropyl dimonium chloride, aloe leaf extract, aloe barbadensis leaf juice, phytantriol, panthenol, retinyl palmitate, behentrimonium methosulfate, cyclopentasiloxane, quaternium-91, stearamidopropyl dimethylamine, and combinations thereof.

Viscosity modifying agents include, for example, viscous liquids, such as polyethylene glycol, semisynthetic polymers, cellulose derivatives, synthetic polymers, naturally occurring polymers, bentonite, colloidal silicon dioxide, and microcrystalline cellulose, and salts, such as sodium chloride, and combinations thereof.

Opacifying agents include, but are not limited to, glycol distearate and ethoxylated fatty alcohols.

In some embodiments, the compositions described herein comprise at least one of a viscosity modifier (e.g., hydoxyethylcellulose or equivalent), a preservative (e.g., phenoxyethanol), an emollient (e.g., propanediol and/or glycerin), a conditioning agent (e.g., behentrimomium methosulfate, argan oil, and/or rosehip oil), or an emulsifier (e.g., cetyl alcohol). In certain embodiments, the composition comprises itaconic acid or a salt, ester, or anhydride thereof, cetyl alcohol, behentrimonium methosulfate, argan oil, rosehip oil, hydroxyethylcellulose, polyquaternium-37, glycerin, propanediol, caprylyl glyceryl ether, and caprylhydroxamic acid. In certain embodiments, the composition comprises itaconic acid or a salt, ester, or anhydride thereof, sodium lauroyl methyl isethionate, sodium C₁₄-C₁₆ olefin sulfonate, hydroxyethylcellulose, guar hydroxypropyltrimonium chloride, glycerin, propanediol, caprylyl glyceryl ether, and caprylhydroxamic acid.

The compositions described herein do not contain any oxidizing agent and are applied to hair fibers without exposure to any oxidizing agent. Exemplary oxidizing agents include UV light in sufficient intensity to initiate chemical oxidation and oxidizing agents commonly used in oxidative hair treatments (such as dying, bleaching, relaxing, and straightening). These include peroxides, oxygenase enzymes, alkali metal bromates or ferricyanides, and peroxygenated salts (e.g., persulfates, perborates, peracids, and precursors thereof as well as percarbonates of alkali metals or alkaline earth metals). Examples of peroxides include hydrogen peroxide and urea peroxide. Examples of persulfates include ammonium, potassium, and sodium persulfates.

In some embodiments, the itaconic acid may be from a petrochemical synthetic source, or may be produced by a fermentation process. Commercially available itaconic acid is primarily from industrial fermentation, using fungi as the fermentation organism. The most common fungi for itaconic acid industrial fermentation are Aspergillus species. In some embodiments, the itaconic acid formulated into cosmetic compositions may be named using the cosmetic nomenclature Aspergillus Ferment or similar nomenclature. Salts, esters and anhydrides of itaconic acid can be suitably prepared by means known in the art.

### Methods of Use

Further aspects of the present invention are directed to methods for treating hair fibers. In general, the methods comprise contacting hair fibers with a composition as described herein. One aspect is directed to a method of treating hair fibers, the method comprising contacting the hair fibers with a hair treatment composition comprising itaconic acid, or a salt, ester, or anhydride thereof, in the absence of any oxidizing agent, wherein the concentration of the itaconic acid or salt, ester, or anhydride thereof in the hair treatment composition is about 0.1% to about 10% by weight.

In various embodiments, the compositions are aqueous compositions and may be applied to the hair by any means suitable (e.g., spraying, dripping, drenching etc.).

In one particular embodiment, hair fibers are treated to reduce flyaway by contacting the fibers with a composition as described herein containing itaconic acid in the absence of any oxidizing agent. For example, the composition described herein can be used to reduce flyaway in over-processed hair fibers such as fibers subjected to repeated bleaching. The itaconic acid-containing compositions can be used in a method to treat hair fibers and reduce flyaway by at least about 10%, at least about 15%, at least about 20%, at least about 25%, at least about 30%, or at least about 35% as compared to hair fibers not treated with a composition containing itaconic acid, salt ester or anhydride thereof.

As used herein the terms “hair” and “hair fibers” to be treated may be “living” (i.e., on a living body) or may be “non-living” (i.e., in a wig, hairpiece or other aggregation of non-living keratinous fibers). Mammalian hair, particularly human hair, is preferred. However, wool, fur, and other keratin containing fibers are suitable substrates for the compositions according to the present invention.

Having described the invention in detail, it will be apparent that modifications and variations are possible without departing from the scope of the invention defined in the appended claims.

### EXAMPLES

The following non-limiting examples are provided to further illustrate the present invention.

### Example 1: Flyaway Test

Five tresses of damaged hair were used to study the impact of itaconic acid on hair flyaway in daily use hair care products, shampoo and conditioner, without the presence of an oxidizing agent. One tress was an untreated control, and four tresses were treated with the four compositions shown in Table 1 and Table 2: shampoo with and without itaconic acid and conditioner with and without itaconic acid.

Tress A was used as the untreated control and was rinsed in water and allowed to dry overnight. Tress B and D were washed with the test shampoos of Table 1, rinsed well, and allowed to dry overnight. Tress C and E was rinsed with water, and the conditioners of Table 2 were applied and allowed to sit for 10 minutes. The tresses were then rinsed thoroughly and allowed to dry overnight.

Materials: Prior to the above treatments, five damaged hair tresses were prepared as follows: A weft of virgin, human, black hair was cut into five 3.8 cm wide strips. The tresses were then each subjected to equal and repeated bleaching in order to create damage in the hair fibers. A 40 volume developer (hydrogen peroxide, L'Oreal Technique Oreore Creme) and a persulfate oxidizing powder (L'Oreal Technique Quick Blue Extra Strength) were applied following the manufacturer's instructions. This was repeated two times, each time for one hour. The damaged hair tresses were washed with a clarifying shampoo and allowed to air-dry overnight. The natural inherent width of each dry damaged tress was 4 cm.

Flyaway Testing: After all five tresses had air dried for 24 hours, they were each brushed exactly 5 times and hung in front of a 2D ruled measuring panel. Tresses were centered on a center line with additional lines marked at 2 cm increments on either side of the tress center line. Hair strands falling outside the 2 cm lines were therefore outside of the inherent 4 cm width prior to brushing. Photographs of the tresses and measuring panel were taken and viewed in the Adobe Photoshop application. The ruler functionality was used to determine horizontal distance of flyaway hair post treatment. Larger tress width corresponds to a greater degree of flyaway.

Calculation: The extent of flyaway is the difference between the horizontal distance before and after brushing. Arithmetically, it is total tress width after brushing minus the 4 cm inherent tress width pre-brushing. The extent of flyaway measurements are presented in Table 3.

The untreated control tress had and extent of flyaway of 6.37 cm. As expected, tresses treated with shampoo and conditioner had less flyaway than untreated control, as shown in Table 3. Unexpectedly, tresses treated with shampoo and conditioner that included itaconic acid (ITA) achieved substantially less flyaway than tresses treated with shampoo and conditioner without itaconic acid.

TABLE 1

| | Shampoo | Shampoo |
|---|---|---|
| | with ITA | without ITA |
| Ingredient | (wt/wt) | (wt/wt) |
| Water | 69.97% | 71.5% |
| Itaconic acid | 1.53% | — |
| Sodium Lauroyl Methyl Isethionate | 5.0% | 5.0% |
| Sodium C₁₄-C₁₆ Olefin Sulfonate | 15.0% | 15.0% |
| Hydroxyethylcellulose | 0.5% | 0.5% |
| Guar Hydroxypropyltrimonium Chloride | 1.0% | 1.0% |
| Glycerin | 5.0% | 5.0% |
| Propanediol | 1.3% | 1.3% |
| Caprylyl Glyceryl Ether | 0.5% | 0.5% |
| Caprylhydroxamic Acid | 0.2% | 0.2% |

TABLE 2

| | Conditioner | Conditioner |
|---|---|---|
| | with | without |
| Ingredient | ITA(wt/wt) | ITA (wt/wt) |
| Water | 74.675% | 78.5% |
| Itaconic acid | 3.825% | — |
| Cetyl Alcohol | 1.0% | 1.0% |
| Behentrimonium Methosulfate | 5.0% | 5.0% |
| Hydroxyethylcellulose | 1.0% | 1.0% |
| Polyquaternium-37 | 0.5% | 0.5% |
| Argan Oil | 1.0% | 1.0% |
| Rosehip Oil | 1.0% | 1.0% |
| Glycerin | 5.0% | 5.0% |
| Propanediol | 6.35% | 6.35% |
| Caprylyl Glyceryl Ether | 0.5% | 0.5% |
| Caprylhydroxamic Acid | 0.15% | 0.15% |

TABLE 3

| | Extent of | Extent of | Flyaway | Flyaway |
|---|---|---|---|---|
| | Flyaway | Flyaway | Improvement | Improvement |
| | without ITA | with ITA | with ITA | with ITA |
| Shampoo | 3.73 cm | 2.88 cm | .85 cm | 22.8% |
| Conditioner | 4.43 cm | 2.53 cm | 1.9 cm | 42.9% |

### Example 2: Half-Head Blind Test

A salon mannequin head with 100% human hair was split into two sections. The sections were both washed with clarifying shampoo and allowed to dry overnight. Once dry, hair was rewet and placebo conditioner was applied to the left side while test conditioner containing itaconic acid was applied to the right side. The conditioner compositions used are shown in Table 2. The conditioners were applied for 10 minutes then rinsed. An initial assessment of the wet hair was performed by 6 trained individuals. These individuals assessed both visual attributes of smoothness, flyaway, and texture as well as tactile attributes of smoothness, combability, and residue feel. The hair was allowed to air-dry overnight, and the same assessment of visual and tactile attributes was performed. All 6 individuals determined the right side was superior in both visual and tactile attributes, reporting that the right side looked and felt smoother, had less flyaway, was easier to comb through, and had less residue feel. They also determined the ends looked less broken.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions, methods, and processes without departing from the scope of the invention, it is intended that all matter contained in the above description shall be interpreted as illustrative and not in a limiting sense.

1. A method of treating hair fibers, the method comprising contacting the hair fibers with a hair treatment composition comprising itaconic acid, or a salt, ester, or anhydride thereof, in the absence of any oxidizing agent, wherein the concentration of the itaconic acid or salt, ester, or anhydride thereof in the hair treatment composition is about 0.1% to about 10% by weight.

2. The method of claim 1, wherein the hair treatment composition comprises itaconic acid.

3. The method of claim 1, wherein the concentration of the itaconic acid or salt, ester, or anhydride thereof in the hair treatment composition is about 0.5% to about 5% by weight.

4. The method of claim 1, wherein the concentration of the itaconic acid or salt, ester, or anhydride thereof in the hair treatment composition is about 1.5% to about 4% by weight.

5. The method of claim 1, wherein the hair treatment composition is a lotion, milk, mousse, spray, gel, or cream.

6. The method of claim 1, wherein the hair treatment composition is a shampoo.

7. The method of claim 1, wherein the hair treatment composition is a rinse-off or leave-on conditioner.

8. The method of claim 1, wherein the hair treatment composition further comprises at least one ingredient selected from the group consisting of preservatives, antioxidants, chelating agents, sunscreen agents, vitamins, dyes, hair coloring agents, proteins, amino acids, natural extracts, humectants, fragrances, perfumes, oils, emollients, lubricants, butters, penetrants, thickeners, viscosity modifiers, polymers, resins, hair fixatives, film formers, surfactants, detergents, emulsifiers, opacifying agents, volatiles, propellants, liquid vehicles, carriers, salts, pH adjusting agents, neutralizing agents, buffers, hair conditioning agents, anti-static agents, anti-frizz agents, anti-dandruff agents, absorbents, and combinations thereof.

9. The method of claim 1, wherein the hair treatment composition further comprises one or more ingredients selected from the group consisting of a viscosity modifier, a preservative, an emollient, a conditioning agent, and an emulsifier.

10. The method of claim 1, wherein the hair treatment composition further comprises cetyl alcohol, behentrimonium methosulfate, argan oil, rosehip oil, hydroxyethylcellulose, polyquaternium-37, glycerin, propanediol, caprylyl glyceryl ether, and caprylhydroxamic acid.

11. The method of claim 1, wherein the hair treatment composition further comprises sodium lauroyl methyl isethionate, sodium C₁₄-C₁₆ olefin sulfonate, hydroxyethylcellulose, guar hydroxypropyltrimonium chloride, glycerin, propanediol, caprylyl glyceryl ether, and caprylhydroxamic acid.

12. A method of treating bleached hair fibers to reduce flyaway, the method comprising contacting the bleached hair fibers with a hair treatment composition comprising itaconic acid, or a salt, ester, or anhydride thereof, in the absence of any oxidizing agent, wherein the concentration of the itaconic acid or salt, ester, or anhydride thereof in the hair treatment composition is about 0.1% to about 10% by weight and wherein the treatment results in at least about 10% reduction in flyaway as compared to bleached hair fibers not treated with a composition containing itaconic acid, or a salt, ester, or anhydride thereof.

13. The method of claim 12, wherein the hair treatment composition comprises itaconic acid.

14. The method of claim 12, wherein the concentration of the itaconic acid or salt, ester, or anhydride thereof in the hair treatment composition is about 0.5% to about 5% by weight.

15. The method of claim 12, wherein the concentration of the itaconic acid or salt, ester, or anhydride thereof in the hair treatment composition is about 1.5% to about 4% by weight.

16. The method of claim 12, wherein the hair treatment composition is a shampoo.

17. The method of claim 12, wherein the hair treatment composition is a rinse-off or leave-on conditioner.

18. The method of claim 12, wherein the hair treatment composition further comprises at least one ingredient selected from the group consisting of preservatives, antioxidants, chelating agents, sunscreen agents, vitamins, dyes, hair coloring agents, proteins, amino acids, natural extracts, humectants, fragrances, perfumes, oils, emollients, lubricants, butters, penetrants, thickeners, viscosity modifiers, polymers, resins, hair fixatives, film formers, surfactants, detergents, emulsifiers, opacifying agents, volatiles, propellants, liquid vehicles, carriers, salts, pH adjusting agents, neutralizing agents, buffers, hair conditioning agents, anti-static agents, anti-frizz agents, anti-dandruff agents, absorbents, and combinations thereof.

19. The method of claim 12, wherein the hair treatment composition further comprises one or more ingredients selected from the group consisting of a viscosity modifier, a preservative, an emollient, a conditioning agent, and an emulsifier.

20. The method of claim 1, wherein the treatment results in at least about 20% reduction in flyaway as compared to bleached hair fibers not treated with a composition containing itaconic acid, or a salt, ester, or anhydride thereof.
